(19) United States
(12) Patent Application Publication
(10) Pub. No.: US 2012/0108495 A1
(43) Pub. Date: May 3, 2012

(54) Method of Inhibiting Smooth Muscle Cell Proliferation by Using Reconstituted HDL

(75) Inventors: Jeong Euy PARK, Seoul (KR); Bok Soo LEE, Seoul (KR); Ki Yong KIM, Gyeonggi-do (KR)

(73) Assignees: GREEN CROSS CORPORATION, Gyeonggi-do (KR); SAMSUNG LIFE PUBLIC WELFARE FOUNDATION, Seoul (KR)

(21) Appl. No.: 13/281,070

(22) Filed: Oct. 25, 2011

(30) Foreign Application Priority Data
Oct. 29, 2010 (KR) 10 2010 0106659

(51) Int. Cl. A61K 38/02; A61P 9/10

(52) U.S. Cl. 514/1.9

(57) ABSTRACT

A method of inhibiting smooth muscle cell proliferation comprising administering to a subject a pharmaceutical composition including reconstituted HDL as an active ingredient.

### CROSS-REFERENCE TO RELATED PATENT APPLICATION

This application claims the benefit of Korean Patent Application No. 10-2010-0106659, filed on Oct. 29, 2010, in the Korean Intellectual Property Office, the disclosure of which is incorporated herein in its entirety by reference.

### BACKGROUND OF THE INVENTION

1. Field of the Invention

The present invention relates to a method of inhibiting smooth muscle cell proliferation including administering a composition including reconstituted HDL, and a blood vessel restenosis animal model.

2. Description of the Related Art

Coronary artery disease is the leading cause of death in the United States, Europe, Russia, and many Eastern European countries, posing a serious health problem, and is considerably increasing in developing countries.

Coronary artery disease is generally caused by deposition of cholesterol on the inner walls of the artery which in turn leads to proliferation of smooth muscle cells and thickening of the walls of the arteries to form atherosclerotic plaques. The disease may advance to acute diseases such as angina pectoris, myocardial infarction, stroke, peripheral artery stenosis, and aortic aneurysm. Theses diseases cannot be completely cured using currently available treatment including antihyperlipidemic agents, and there is the risk of recurrence and progress of the diseases. Among the available treatments for vascular diseases including atherosclerotic coronary artery disease are drugs such as statins that lower the levels of low density lipoprotein (LDL) and very low density lipoprotein (VLDL) and stabilize atherosclerotic lesions, and help preventing the progression of atherosclerosis. As a surgical treatment, artery bypass surgery has widely been used. A long-term administration of statins for more than 4 years may reduce the LDL level by 50% or more and death caused by coronary arterial events and stroke by about 25 to 30%. However, 70 to 75% of the patients still have the risk of recurrence and progress of atherosclerotic cardiovascular disease. Thus, there is a need to develop drugs for preventing and treating arteriosclerotic vascular diseases and blood vessel restenosis.

The inventors conducted research to develop drugs capable of inhibiting the progression of atherosclerotic lesions or preventing the intimal hyperplasia and restenosis after balloon injury.

### SUMMARY OF THE INVENTION

The present invention provides a method of inhibiting neointimal thickening by inhibition of smooth muscle cell proliferation, the method including administering a composition including reconstituted HDL as an active ingredient.

The present invention also provides a method of preventing or treating atherosclerotic coronary artery disease or blood vessel restenosis, the method including administering a composition including reconstituted HDL as an active ingredient.

The present invention also provides a blood vessel restenosis animal model.

### BRIEF DESCRIPTION OF THE DRAWINGS

The above and other features and advantages of the present invention will become more apparent by describing in detail exemplary embodiments thereof with reference to the attached drawings in which:

FIG. 1 shows photographs illustrating the effect of infusion of reconstituted HDL (rHDL) on injured rat carotid arteries according to an embodiment of the present invention, wherein the rHDL infusion was performed at a dose of 80 mg/kg at 4 hours before surgery and at 24 hours and 48 hours after surgery. In this figure, (a), (c), and (e) show the status of restenosis at 4 days after surgery and (b), (d), and (f) show the status of restenosis at 4 weeks after surgery;

FIG. 2 shows a graph indicating that the infusion of rHDL according to an embodiment of the present invention reduces restenosis;

FIG. 3 shows that the infusion of rHDL according to an embodiment of the present invention reduces neointimal hyperplasia; FIGS. 3A and 3B shows the results of immunostaining against PCNA and Ki-67, respectively.

FIG. 4 shows graphs indicating that the infusion of rHDL according to an embodiment of the present invention significantly induces endothelial cell proliferation but inhibits smooth muscle cell proliferation; FIGS. 4A and 4B show the proliferation of endothelial cells(EPC) and vascular smooth muscle cells(VSMC), respectively.

FIG. 5 shows photographs indicating that the infusion of rHDL according to an embodiment of the present invention significantly reduces monocyte infiltration;

FIG. 6 shows photographs indicating that the infusion of rHDL according to an embodiment of the present invention reduces late loss of the in-stent restenosis, wherein FIGS. 6A, 6B and 6C show the degree of restenosis in rabbit models fed with a normal chow, a high fat high cholesterol (HFHC) chow, and a HFHC chow+HDL, respectively; and

FIG. 7 shows a photograph (FIG. 7A) and a graph (7B) indicating that the infusion of rHDL according to an embodiment of the present invention reduces late loss of the in-stent restenosis, wherein the normal, HDL X, and HDL O respectively refer to a group fed with a normal chow, a group not treated with HDL (HDL X), and a group treated with HDL (HDL O).

### DETAILED DESCRIPTION OF THE INVENTION

Hereinafter, exemplary embodiments of the present invention will be described in detail with reference to the accompanying drawings.

According to an aspect of the present invention, provided is a method of inhibiting neointimal thickening by inhibition of smooth muscle cell proliferation, the method including administering a composition including reconstituted high density lipoprotein (rHDL) as an active ingredient.

The rHDL used herein refers to HDL that is reconstituted using apolipoprotein A-I (ApoA-I), phospholipid, and a surfactant.

According to an embodiment of the present invention, the rHDL includes ApoA-I and phospholipid such as phosphatidylcholine, wherein the molar ratio of ApoA-I to phospholipid may be in the range of 1:50 to 1:250. In addition, the rHDL may further include lipid such as cholesterol.

A main function of the HDL in living organisms is a reverse cholesterol transport (RCT) by which cholesterol is reversely transported from tissues to liver to excrete the cholesterol via bile, and the HDL is involved in the lipid metabolism in blood as a carrier of cholesterol, neutral fats, or the like. In addition, the HDL has diverse functions contributing to the stabilization, inhibition of the progression, or sometimes even regression of atherosclerotic lesion. For example, the HDL, inhibits the formation of atherosclerotic plaques by promoting excretion of residual cholesterol from peripheral cells, activating relevant enzymes, and inhibiting oxidation of LDL. That is, as well as the reverse cholesterol transport, the HDL also has anti-atherogenic, anti-oxidant, anti-inflammatory, anti-thrombotic, and anti-infectious activities. Furthermore, the HDL contributes to enhanced function of vascular endothelial cells, increase in the number of vascular endothelial progenitor cells, and improved insulin secretion capability of pancreatic beta-cells.

When the rHDL is administered to animals having blood vessel restenosis induced by balloon-injured artery or a stent insertion, smooth muscle cell proliferation has been found to be inhibited. The rHDL may be used to stabilize arteriosclerotic lesions and relieve or prevent thrombus. In addition, the rHDL may reduce the degree of injury of myocardium at an early stage of treatment in a patient with the acute coronary syndrome, stabilize atherosclerotic plaques, and inhibit hyperplasia of vascular endothelial cell when balloon angioplasty is performed or a stent is inserted, thereby contributing to the prevention of blood vessel restenosis.

The smooth muscle cells in blood vessels proliferate in response to injury to the blood vessel walls. The proliferation occurs significantly in atherosclerosis showing secondary changes in endarteriums from the lipid induced injuires to the blood vessel walls. Thus, the smooth muscle cell proliferation has been known as a main cause of atherosclerosis and restenosis.

According to an embodiment of the present invention, the composition may inhibit neointimal hyperplasia in blood vessel.

Endothelium or intima of blood vessels separates blood from peripheral tissues thereof and controls important functions of blood vessel walls. Integrity in tissue and action is an Important precondition for complete function of a layer of endothelial cells. The endothelial cells control adhesion of cells such as monocytes and platelet, infiltration of immunocytes, and proliferation of smooth muscle cell and generate bioactive substances such as nitrogen monoxide or endothelin-1. If endothelial cell proliferation is induced in response to injury of blood vessels, and accordingly neointima thickens, functions of endothelium may deteriorate to promote the progression of atherosclerosis. The infusion of rHDL may inhibit neointimal hyperplasia in blood vessels so that the progression of atherosclerosis may be inhibited or prevented.

According to another aspect of the present invention, provided is a method of preventing or treating atherosclerotic coronary artery diseases or blood vessel restenosis, the method including administering a composition including rHDL as an active ingredient.

As described above, the vascular smooth muscle cell proliferation is a main cause of atherosclerosis. Thus, the atherosclerotic coronary artery diseases may be prevented or treated by inhibiting the vascular smooth muscle cell proliferation.

While coronary artery stenosis caused by atherosclerosis slowly occurs for several years, restenosis thereof occurs within several weeks or several months as a result of various molecular biological and cell biological chain reactions in blood vessel walls caused by expansion of coronary artery. Thus, pathophysiological characteristics of blood vessel restenosis are different from those of general atherosclerosis. Artificial mechanical injuries are caused to blood vessel walls during coronary artery intervention, and accordingly, a number of vasoactive substances, thrombus generating substances, and mitogens are secreted as a natural recovering process, leading to vigorous cellular reactions. As a result, neointimal hyperplasia occurs to narrow lumen of blood vessels, and thereby causing blood vessel restenosis in every direction. According to recent research, most of neointimal hyperplasia includes the following two phases [Tung R., et al., Narrative review: drug-eluting stents for the management of restenosis: a critical appraisal of the evidence. Ann. Intern. Med. vol. 144(12):913-919(2006)]. The first phase is an inflammation phase. In this phase, blood vessels injured by balloon angioplasty or stent insertion have local thrombus formation as a natural healing process, and accordingly inflammatory cells vigorously infiltrate into vascular endothelium. The infiltration of inflammatory cells occurs principally between 3 to 7 days after surgery. The second phase is a proliferative phase. In this phase, the infiltrated inflammatory cells stimulate vascular smooth muscle cells, leading to that their proliferation. This occurs usually after 1 week from the surgery.

Thus, blood cell restenosis may be prevented or treated by administering rHDL which can relieve inflammation of the smooth muscle cells, and inhibit their proliferation.

Hereinafter, the present invention will be described in more detail with reference to the following examples. However, these examples are not intended to limit the scope of the one or more embodiments of the present invention.

### EXAMPLES

### Example 1

### Effect of rHDL on Inhibition of Restenosis in Rats

1-1. Induction of Blood Vessel Restenosis by Balloon Arterial Injury

Ketamine and xylazine were subcutaneously injected into a 6 week-old male Sprague-Dawley rat (Jackson Laboratories) to anesthetize the rat. The right carotid artery of the rat was incised and exposed, and the incised muscles were fixed such that arteries were visible. A 2F Fogarty catheter (Edwards Lifesciences) was inserted into the external carotid artery and pushed into the common carotid artery (common CA), and ballooned. The endothelium was artificially denuded by moving the catheter back and forth 3 times. The catheter was removed and the external carotid artery was tied in order to stop bleeding, and then the incised portion was sutured. When awakened, the rat was returned to a cage. 40 mg/kg or 80 mg/kg of rHDL was administered to a rHDL treated group at 4 hours before surgery, and at 24 hours and 48 hours after surgery. After 4 weeks from the surgery, the injured carotid artery was fixed in 10% neutralized formalin and embedded in a paraffin block. Then, 4 μm thick sections of the carotid artery were prepared and stained with hematoxylin and eosin (H&E). The areas of neointima and media were measured and the ratio thereof was calculated to determine the degree of inhibition of blood vessel restenosis.

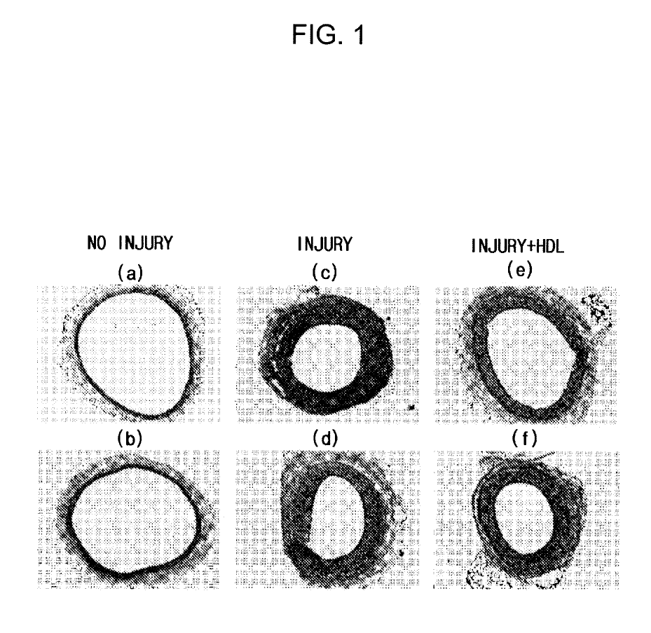

FIG. 1 shows carotid arteries stained with H&E. When the rHDL is administered, the thickness of media of blood vessel was found to be well preserved.

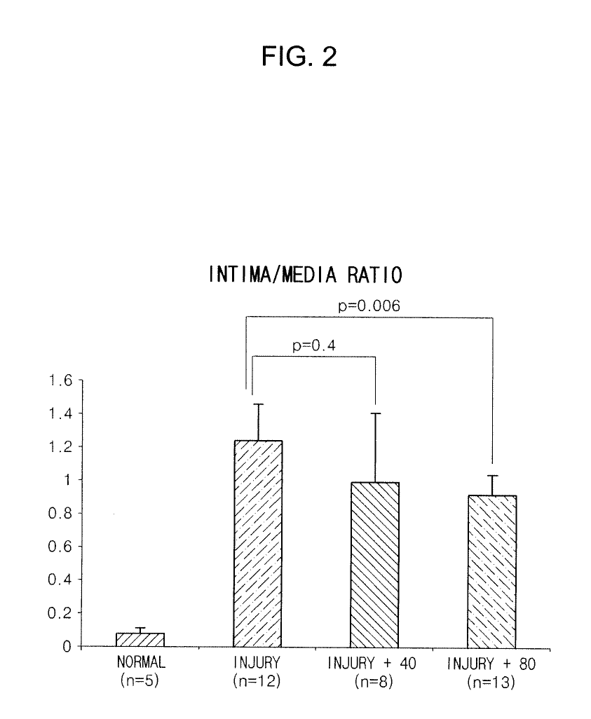

Referring to FIG. 2, the ratio of intima/media that is a reference of the restenosis of blood vessel is reduced depending on the concentration of the rHDL. Change in the thickness of the intima, rather than that of the media was found to be the main cause of the change in the ratio.

1-2. Neointimal Hyperplasia

Blood vessel restenosis occurs via hyperplasia of injured vascular cells. In order to identify the effect of rHDL on the inhibition of restenosis of blood vessel, the tissue sections obtained in the above 1-1 were immuno-stained with proliferating cell nuclear antigen (PCNA) (PC-10, Cell Signaling, Danvers, Mass.) showing the degree of cell division and Ki-67 antibody (SP6, Novus, Colo., USA) (J. EthnoPharmacol. 2010, Jo Yoon Yi, Lee et al., In press).

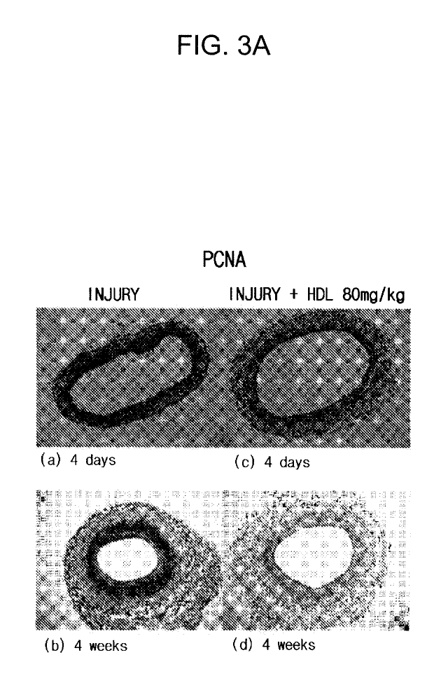

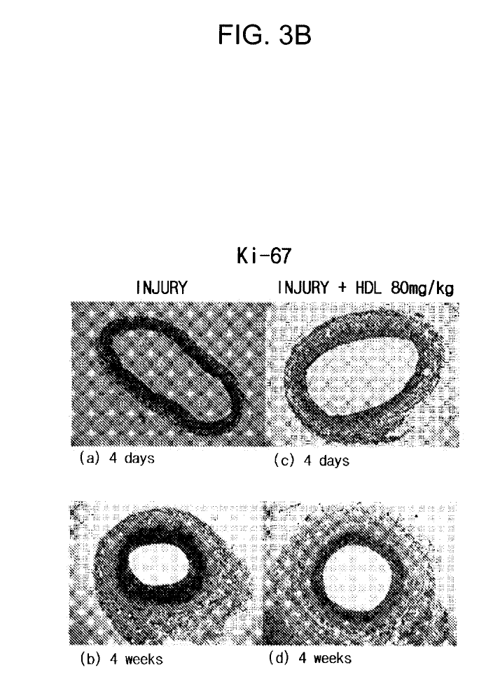

FIG. 3 shows stained sections at 4 days after surgery (the photographs in the upper section), and at 4 weeks after surgery (the photographs in the lower section). In an rHDL treated group, neointimal hyperplasia was inhibited.

1-3. Effect of rHDL on Endothelial Cell Proliferation and Smooth Muscle Cell Proliferation

Vascular endothelial progenitor cells were prepared by isolating monocytes from human umbilical cord blood, growing them in a culture medium in which vascular endothelial progenitor cells selectively grow for 2 to 4 weeks, and selecting cells expressing vascular endothelial progenitor cell markers. Smooth muscle cells were prepared by denuding endothelial cells and adventitia of umbilical cord blood vessel and isolating single smooth muscle cells with an enzyme mixture solution including collagenase. Cultured cells were pre-treated with HDL for 1 hour and treated respectively with a vascular endothelial growth factor (VEGF) and a platelet-derived growth factor (PDGF) for 48 hours.

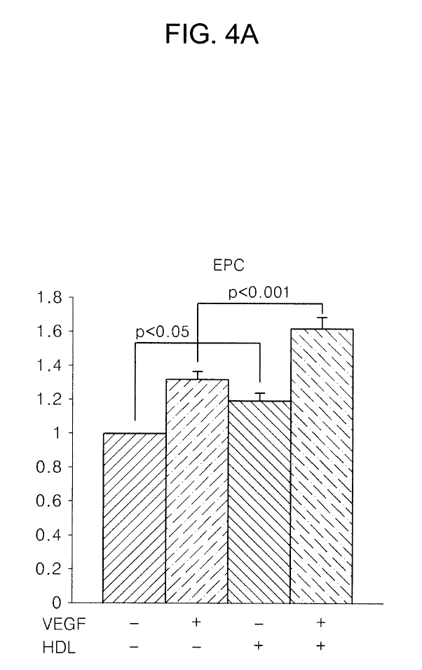

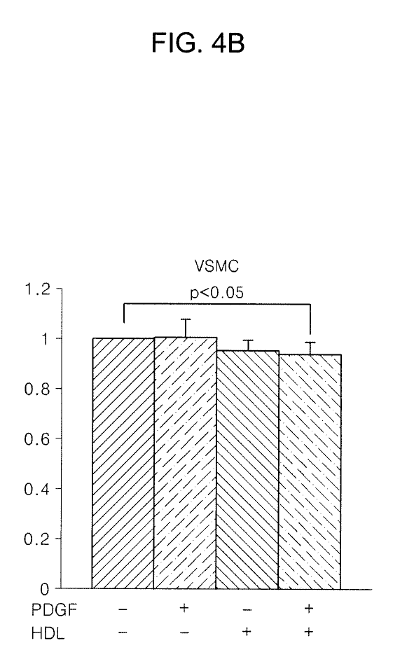

FIG. 4 shows that rHDL significantly induces endothelial cell proliferation, but inhibits smooth muscle cell proliferation.

1-4. Effect of rHDL on Infiltration of Immune Cells

HDL is known to have anti-inflammatory effect. In order to investigate effect of rHDL on infiltration of immune cells into injured blood vessels, the expression level of CD68 on the surface of monocytes/macrophages was measured using an immuno-staining.

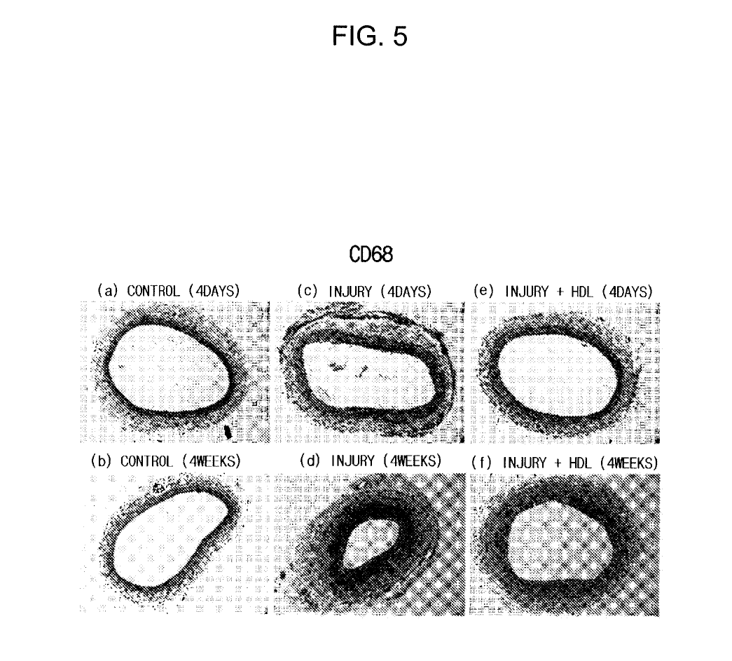

Referring to FIG. 5, rHDL significantly reduces infiltration of monocytes.

### Example 2

### Effect of rHDL on Inhibition of Blood Vessel Restenosis in Rabbits

2-1. Building In-stent Restenosis Model

Ketamine and xylazine were subcutaneously injected into a 3 kg New Zealand White rabbit to anesthetize the rabbit. The carotid artery of the rabbit was partially incised and exposed, and the incised muscles were fixed such that arteries were visible. A guide catheter was inserted into iliac artery of the rabbit using a guide wire under X-ray fluoroscopy, a stent mounted on a balloon was positioned in the iliac artery through the guide catheter, and the stent was safely installed in the iliac artery by expanding the balloon by applying 6 atm. or more thereto. Then, a safe insertion of the stent was verified by using an angiography device. In this regard, pressure applied to the balloon expansion was adjusted such that the diameter of the blood vessel became 1.5 times its original diameter under X-ray fluoroscopy. Then, rabbits with the stents inserted were divided into three groups, each including 11 rabbits and the groups were respectively fed with a normal chow, a high fat high cholesterol (HFHC) chow, and a HFHC+rHDL (ApoA-Lphosphatidylcholine=1:4). After 4 weeks, the stent-inserted portion of the iliac artery was collected, lesions were identified using optical coherence tomography (OCT), and tissues were fixed. Areas of neointima, media, and lumen were measured to determine the degree of inhibition of blood vessel restenosis.

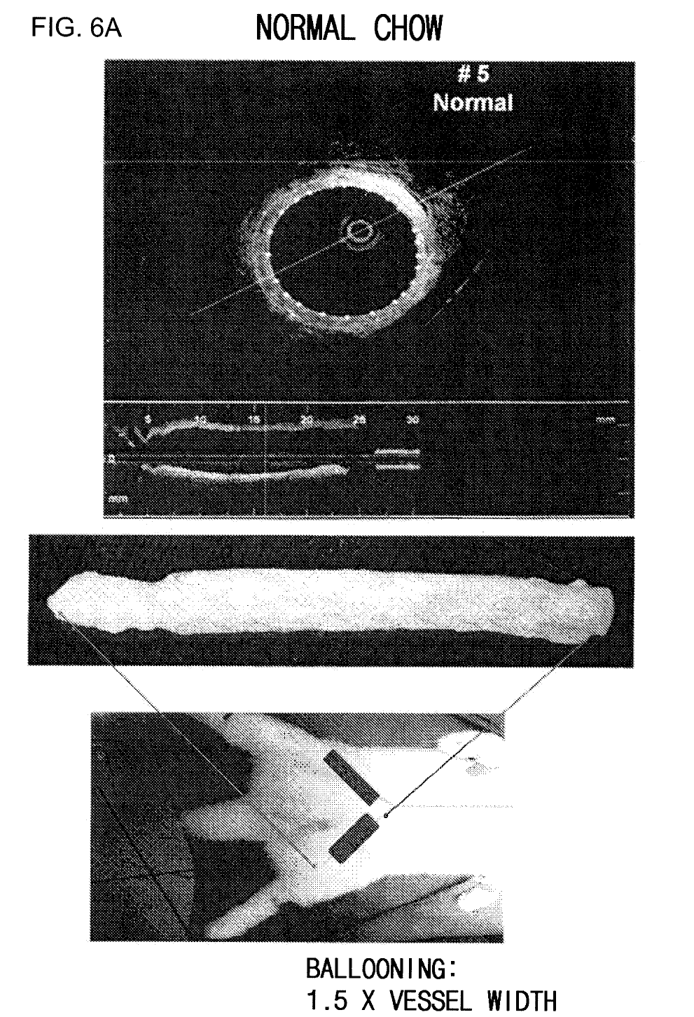

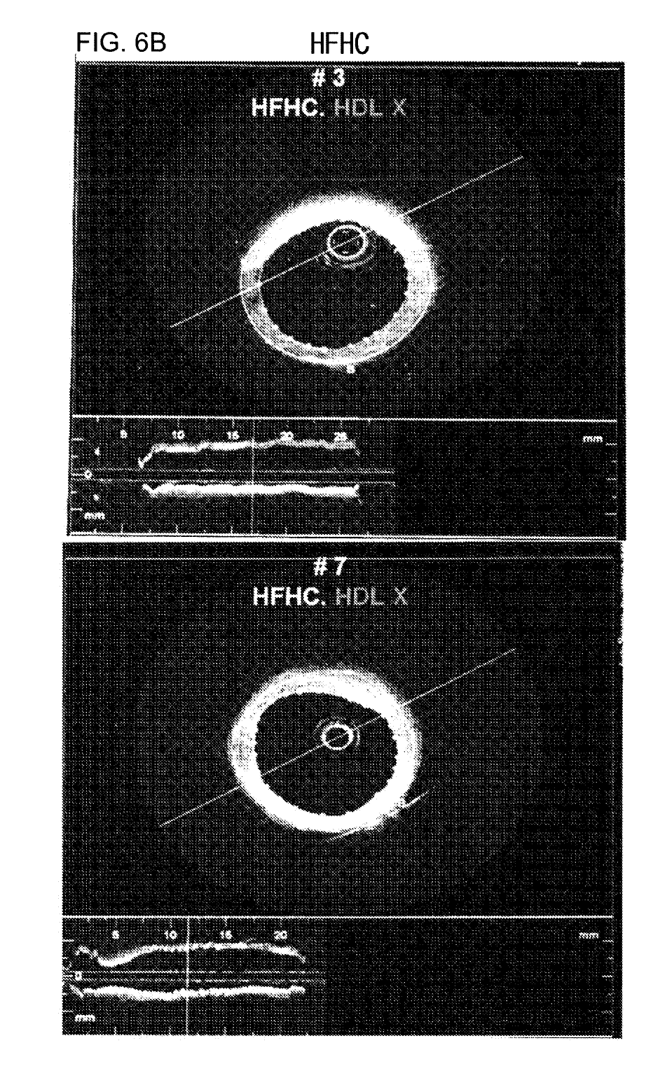

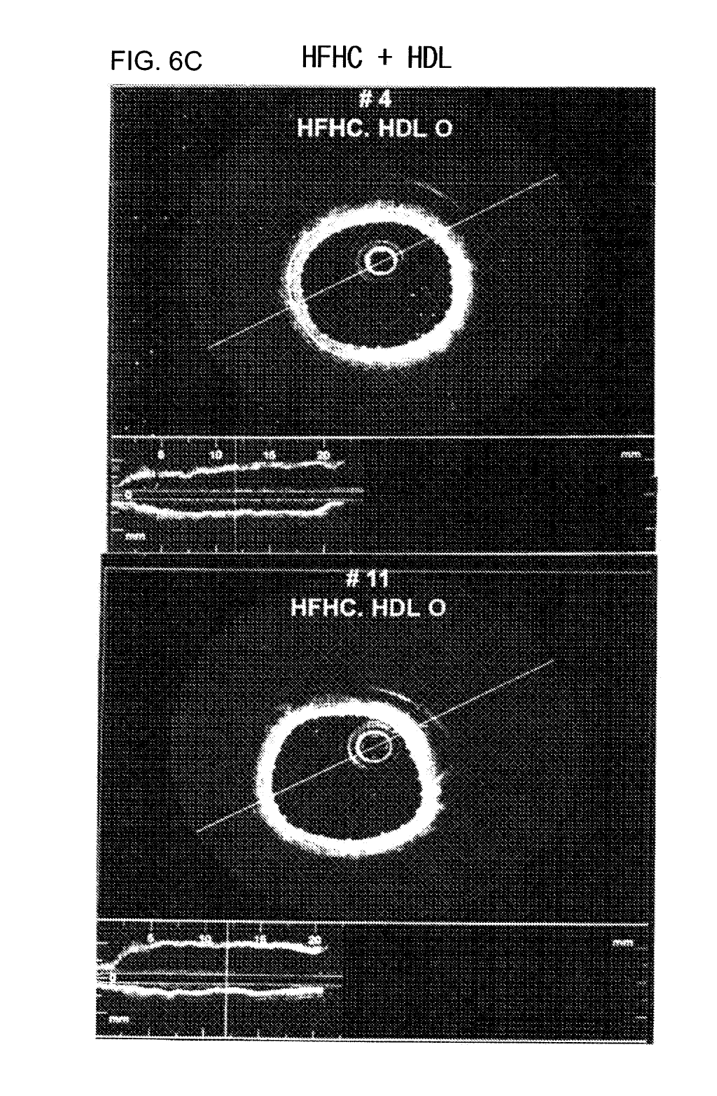

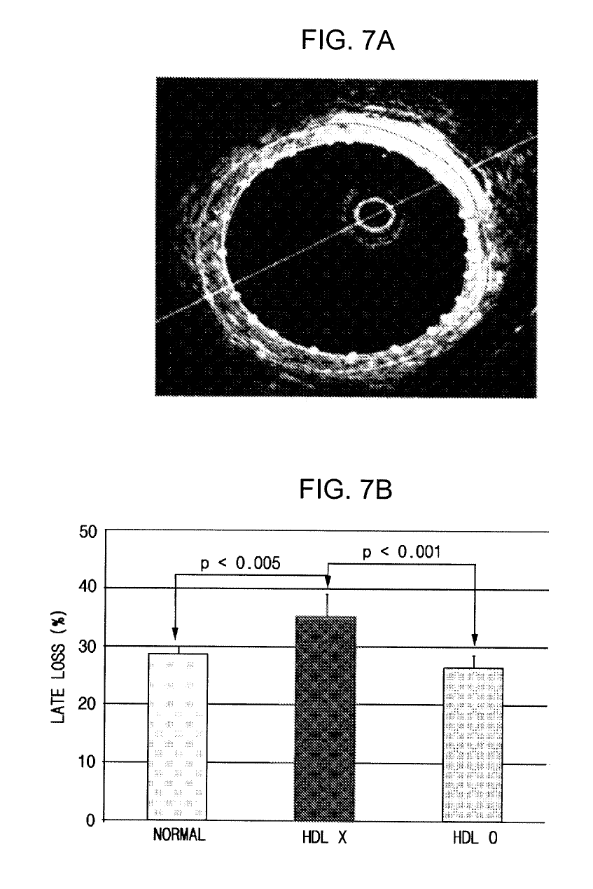

Referring to FIGS. 6 and 7, rHDL reduces late loss of in-stent restenosis. The late loss was calculated by the following formula:

$$[ \frac{( inner strut area ) - ( inner lumen area )}{inner strut area} ] per a frame \times 10 frames \times 100 ( \% )$$

wherein each frame is taken with a 1 mm interval.

2-2. Measurement of Area of Atherosclerotic Lesion Induced by HFHC Chow

As described in the above 2-1, 8 week-old rabbits having a high risk of developing atherosclerosis by HFHC chow were used. The rabbits fed with HFHC chow for 2 weeks were used as a positive control group, and rabbits fed with a HFHC+HDL chow with HDL administered twice a week for 4 weeks using intravenous infusion (i.v.) were used as a test group. After the rabbits were fed with HFHC chow for 10 weeks, blood was collected therefrom, and the rabbits were sacrificed. Then, heart, ascending aorta, aortic arch, and descending thoracic aorta were excised, collected and fixed in formalin. The heart was hardened, cut, embedded in an optical cutting temperature (OCT) complex, and lyophilized in a cryogenic freezer. Then, aortic arch was sliced to a thickness of 5 μm using a cryostat slicer to measure areas of atherosclerotic lesions.

It was identified that the area of arteriosclerotic lesions was reduced in the rHDL treated group.

As described above, the composition including rHDL as an active ingredient according to an embodiments of the present invention may be efficiently used to prevent or treat atherosclerotic coronary artery disease and restenosis of blood vessel by inhibiting smooth muscle cell proliferation in blood vessels.

While the present invention has been particularly shown and described with reference to exemplary embodiments thereof, it will be understood by those of ordinary skill in the art that various changes in form and details may be made without departing from the spirit and scope of the present invention as defined by the following claims.

What is claimed is:

1. A method of inhibiting neointimal thickening by inhibition of smooth muscle cell proliferation, the method comprising administering a composition comprising reconstituted HDL as an active ingredient.

2. The method of claim 1, wherein the composition inhibits neointimal hyperplasia.

3. The method of claim 1, for prevention or treatment of atherosclerotic coronary artery disease or restenosis of blood vessel.
